(19) United States
(12) Patent Application Publication
(10) Pub. No.: US 2014/0336949 A1
(43) Pub. Date: Nov. 13, 2014

(54) METHOD, APPARATUS, AND KIT FOR ANALYZING GENES

(71) Applicant: Hitachi, Ltd., Tokyo (JP)

(72) Inventors: Takahide Yokoi, Tokyo (JP); Takashi Anazawa, Tokyo (JP)

(21) Appl. No.: 14/122,680

(22) PCT Filed: Oct. 19, 2012
(86) PCT No.: PCT/JP2012/077029

§ 371 Date: Nov. 27, 2013

(51) Int. Cl. C12Q 1/68; G06F 19/22

(52) U.S. Cl. CPC C12Q 1/6869; G06F 19/22
USPC 702/20; 435/6.1

(57) ABSTRACT

The conventional DNA sequencers for analyzing nucleotide sequences have no function of detecting minute polymorphisms. Any cross talk in the wavelengths of fluorescent substances for labeled DNA fragments hinders detection of weak-strength signals at the same coordinates, making it difficult to detect genetic mutations with small existence ratios, for example, in somatic mutations. Disclosed is a gene analyzer composed of a plurality of flow channels, each of which is used to electrophorese nucleic acid samples labeled for each of nucleotide types; a chromatogram data creating part for detecting a labeled signal for each of the nucleotide types for each of the nucleic acid samples in each of the plurality of flow channels and creating chromatogram data on signal strengths detected; a peak detection part for the peal values in the chromatogram data for each of the nucleotide types; and a data integrating part for integrating a plurality of chromatogram data.

### TECHNICAL FIELD

The present invention relates to a method, kit, and system for analyzing genes. In particular, the present invention relates to a technique for detecting gene polymorphisms contained in a target gene region.

### BACKGROUND

DNA is a polymer molecule, which carries biogenetic information, and DNA sequencing techniques have enormously evolved as techniques preferable for life science since the Sanger method was developed as one of the methods for analyzing DNA sequences. The “dideoxy method”, a technique developed by Sanger et al., is a sequencing method using synthetic reactions that stop at the positions, at which dideoxynucleotides (ddATP•ddGTP•ddCTP•ddTTP) are uptaken when added in a DNA synthetic reaction solution as low concentration of terminators.

The initial DNA sequencing technique labels four types of dideoxynucleotides with their corresponding radioisotopes; induces a DNA synthetic reaction separately in four vessels, each being filled with the corresponding radioactively-labeled dideoxynycleoride; separates the products of each DNA synthetic reaction based on the lengths of DNA fragments by acrylamide gel electrophoresis using the individual lanes; and determine the nucleotide sequences by detecting the positions, at which radioactive isotopes are found, using autoradiography.

After then, a nucleotide identification method, which uses four kinds of fluorescent dyes, each corresponding one of four types of dideoxynucleotides, has been developed to detect four types of nucleotides together in a mixture thereof, making it possible to analyze DNA sequences on single lane of an acrylamide gel. Moreover, capillary electrophoresis has been developed as a successor electrophoretic method to acrylamide gel electrophoresis. A DNA sequencer using capillary electrophoresis is a system for analyzing one DNA sample labeled with four fluorescent dyes in a single capillary using the Sanger method. A DNA sequencer using capillary electrophoresis, which enables a plurality of samples to be quickly analyzed simultaneously for continuous automatic analysis, have contributed greatly to the large-scale gene sequencing project of, for example the human genome project, of which completion was reported in 2003, and is most widely used at present.

The principle of the DNA sequencer to determine the gene sequences of target samples involves the steps of separating the DNA fragments based on their fragment lengths by electrophoresis and detecting the fluorescent-labeled molecules at the separation positions. The nucleotides are deduced at the coordinate positions, at which their signal peaks are detected, by the rule of majority, based on the strengths of obtained fluorescent signals or the areas of the peaks.

It is known that the genomes of most of living organisms including human being, who are the most preferable target to analyze the gene sequences, are diploid and their genome sequence has mutations, called single nucleotide polymorphisms. These nucleotide polymorphisms, also called germ line polymorphisms, have properties transmitted from parent to offspring and exist in the individuals or cells in a one to one ratio. When the region, in which these nucleotide polymorphisms exist, is analyzed by a DNA sequencer, two kinds of fluorescent peaks are detected simultaneously at the positions, at which the nucleotide polymorphisms are found.

As mentioned before, since the germ line polymorphisms exist in a one to one ratio, basically, these two kinds of fluorescent signals are also detected at the positions, at which germ line polymorphisms are found in a one to one ratio. However, the strengths of signals may not show the one to one correlation at the some nucleotide positions due to a difference in luminescent efficiency between fluorescent substances or a difference in uptaking efficiency between the positions, at which polymorphisms are found; accordingly, the conventional DNA sequencing techniques have a disadvantage of difficulty in detection of the polymorphisms.

To address this problem, a specialized method for analyzing the obtained fluorescent signals to detect these polymorphisms has been developed (Japanese Unexamined Patent Application Publication No. 2002-05508). Furthermore, a mobility shift may occur between the peak positions of chromatograms when a difference in luminescent efficiency between fluorescent substances affects the mobility during electrophoresis. A method for determining the polymorphisms, of which peak positions shifted, has been also developed (Japanese Unexamined Patent Application Publication No. 2003-270206).

Japanese Unexamined Patent Application Publication No. 2002-05508 and Japanese Unexamined Patent Application Publication No. 2003-270206 disclose methods for enhancing the determination accuracy and sensitivity of polymorphisms by reference to existing chromatogram datas in a target gene region. These methods provide effective tools for determining the gene sequences including germ line polymorphisms by analyzing existing datas from the sequencer with a high degree of accuracy.

### SUMMARY

With recent evolution of genome analysis techniques, the DNA sequence of the entire human genome was reported in 2003, and since then, drug development has been proactively advanced taking advantage of gene information. In particular, medical insurance has been applied to genetic testing conducted on the individual patients with cancers, which may be caused by genetic abnormalities, to select some therapeutic medicines and determine the dosages of these medicines.

Genetic disruptions induced by diseases, such as cancers, are called somatic mutations, differently from the aforementioned germ line polymorphisms. The somatic mutations, which are genetic abnormalities occurred after birth, are characterized in that they are not transmitted from parent to offspring, the positions on the genome, in which mutations have occurred, cannot be estimated, and the existence ratio of polymorphisms cannot be estimated in vivo or tissues. Inability to estimate the existence ratio of somatic mutations has become a major problem in detecting these polymorphisms. Giving an example, the cancer tissue excised out from a cancer patient contains both cancer cells and normal cells, and diversity in genetic abnormality is observed among cancer cells, leading to a low existence ratio of the cells having polymorphisms in the target region of the tissue. For this reason, detection of somatic mutations is more difficult than that of germ line polymorphisms.

Currently, quantitative polymerase chain reaction (PCR) system and DNA sequencers are used for detecting somatic mutations. The quantitative PCR system has a great advantage of the high-sensitivity detection. However, it also is a disadvantage in that a specific detection probe for each target polymorphism is necessary to conduct the detection of reactions using separate probes. In particular, for cancer cells, what types of genetic abnormalities may occur and where cannot be estimated at present; accordingly, any quantitative PCR system, for which probe design specific for the target polymorphism is needed, is not suitable for exhaustive detection of somatic Mutations. Even if a various kinds of combinations of detection probes are available, it would be practically difficult to detect somatic mutations using all these probes due to limited testing cost and analyte samples in amount.

On the other hand, a capillary electrophoresis-based DNA sequencer, the most widely used sequence system at present, has an ability to determine 500-700 bp. For this reason, this type of sequencer has advantages over the quantitative PCR system in that it is capable of 1) detecting a larger region in initial testing, and ii) determining new somatic mutations in the aforementioned nucleotide sequence region. In addition, the quantitative PCR system determines gene sequences based on the relative intensity between signal values for the target polymorphism, while the DNA sequencer, can verify that the genetic polymorphism detected using target polymorphism information is derived truly from the target gene, resulting in highly reliable results of measurement compared with those of the quantitative PCR system. High reliability of measurements is one of preferable features in medical diagnosis, in which accurate determination is needed. However, the DNA sequencer, the system specialized for gene sequencing, has a major problem of insufficient sensitivity to detect minute somatic mutations.

In contrast, the capillary electrophoresis-based DNA sequencer is capable of determining nucleotide sequences using four kinds of fluorescent labeling substances, each corresponding to one of four types of nucleotides. Generally, the fluorescent substance produces luminescence across a wide range of wavelengths but not at a single wavelength. FIG. 2 shows the wavelength ranges of four kinds of general-purpose fluorescent substances used in nucleotide sequence analysis. These four kinds of fluorescent substances have different peak wavelengths (2a -2d), but each has a cross talk as known from FIG. 2. The DNA sequencer involves the steps of separating the labeled DNA molecules based on their DNA fragment lengths by electrophoresis; measuring the strengths of the signals across the peak wavelength ranges; and identifying main fluorescent signals to determine the nucleotide sequence; accordingly, the existence of the aforementioned cross talk has not become a major problem for such an intended use. However, to detect DNA molecules having somatic mutations with the existence ratio less than one to one, the existence of the cross talk prevents the signals generated by a minute amount of DNA molecule at the position of same DNA fragment length from being detected, leading to a deterioration in sensitivity of genetic mutations detection. Both the methods, which are disclosed in the aforementioned Japanese Unexamined Patent Application Publication No. 2002-05508 and Japanese Unexamined Patent Application Publication No. 2003-270206, use the values for the signals output from the sequencer as input information; thereby, they could not improve sufficiently the deteriorated sensitivity of genetic mutation detection due to the existence of cross talks, which in turn, has prevented genetic mutations of somatic mutations to be detected sufficiently.

According to one aspect of the present invention, to solve at least one of the aforementioned problems, the nucleic acid samples are labeled for each of nucleotide types; the labeled samples are electrophoresed in the separate flow channel for each of nucleotide types; and genetic mutations are detected based on chromatogram data obtained from the labeled signal for each of nucleotide types concerning the individual nucleic acid samples separated by electrophoresis in its corresponding one of the plurality of flow channels.

The present invention enables the somatic mutations existing in the target gene region to be detected with a high order of sensitivity. The problems and configurations other than those above mentioned will be explained using the following embodiments.

### BRIEF DESCRIPTION OF THE DRAWINGS

FIG. 1 is a diagram showing the configuration of a measuring apparatus using individual flow channels according to an embodiment of the present invention;

FIG. 2 is a diagram showing the wavelengths of signals labeled with four different fluorescent dyes, which are generally used for DNA sequencing;

FIG. 3 is a flow sheet illustrating the steps of detecting peaks; correcting the mobility; and integrating data from flow channels according to one embodiment of the present invention;

FIG. 4 is a flow sheet illustrating the steps of detecting peaks; correcting the mobility; and integrating data from the flow channels according to another embodiment of the present invention;

FIG. 5 is a flow sheet illustrating the steps of detecting peaks; correcting the mobility; and integrating data from the flow channels according to further another embodiment of the present invention;

FIG. 6 is a flow sheet illustrating the steps of detecting peaks; correcting the mobility; and integrating data from the flow channels according to other embodiment of the present invention;

FIG. 7 is a view showing the representation of the integrated chromatogram data and information on detected polymorphisms according to the embodiment of the present invention;

FIG. 8 is a diagram showing the configuration of an entire system according to the embodiment of the present invention; and

FIG. 9 is a block diagram showing the configuration of functions, which a data analyzer has, according to the embodiment of the present invention.

### DETAILED DESCRIPTION

Hereinafter, an embodiment of the present invention will be explained by reference to the accompanying drawings. It should be noted that the exemplified embodiments of the present invention include but not limited to those explained below.

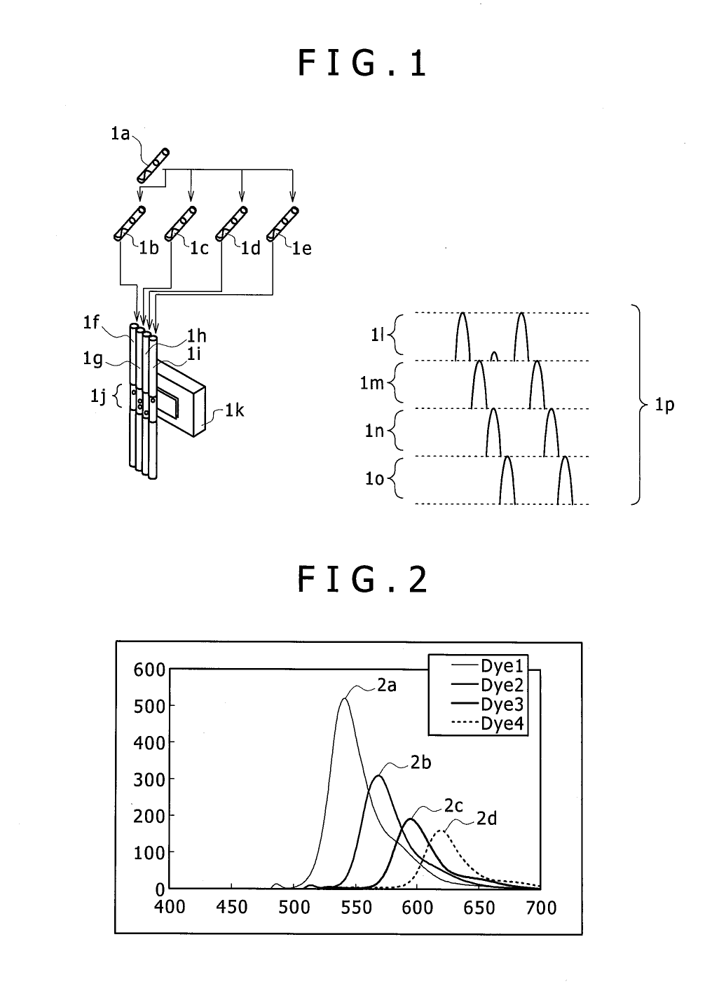

First, the method for detecting DNA molecules according to an embodiment of the present invention will be explained by reference to FIG. 1. The method is initiated by preparing a target DNA (template DNA) sample 1a. Generally, the template DNA is amplified by the polymerase chain reaction (PCR) method, which amplifies specifically a target genetic region but according to the embodiment of the present invention, the method for preparing the template is not limited to the PCR method.

A primer having a complimentary sequence in part of the template DNA and DNA synthetase, as well as dNTP and ddNTP as reactive substrates, are added in a solution containing the template DNA sample (1a) to induce a labeling reaction by the Sanger method. According to the embodiment of the present invention, a pair of the template DNA sample and the primer is labeled with four different reactive solutions (1b-1e) corresponding to the individual target nucleotide types (A,G,C,T) to separate these target nucleotide types (A,G,C,T) in the separate flow channels (1f-1i) by electrophoresis and make measurement.

According to the embodiment of the present invention, either 1) the dye primer method, which labels the primer i), or 2) the dye terminator method, which labels ddNTP ii), may be used for labeling synthetic DNA molecules. Moreover, any of fluorescent dyes, chemical luminescent substances, and radioactive isotopes may be used as labeling substances. The sequencing kits commercially available today label four types of ddNTPs with different fluorescent substances; they may be applied to the labeled DNA sample containing four types of ddNTPs labeled with different fluorescent substances according to the embodiment of the present invention. Either the dye primer method or the dye terminator method may be used, as the labeling method, because four types of ddNTPs labeled with a single kind of fluorescent substance are analyzed in the physically separate flow channels by electrophoresis.

In particular, with the dye terminator method, when ddATP, ddGTP, ddCTP, and ddTTP are labeled with different fluorescent substances, differences in chemical structure among these different fluorescent substances affect the uptaking efficiency during labeling reaction. For this reason, labeling the four types of nucleotides with a single kind of fluorescent substance is useful in detecting the existence ratio correctly. Moreover, the differences among the fluorescent substances also affect the mobility of DNA fragments during electrophoresis; accordingly, labeling the four types of ddNTPs with the single fluorescent substance is also useful in correcting the mobility of the DNA fragments.

Second, the labeled DNA fragments are separated in the separate flow channels (1f - 1i) for each of four nucleotide types by electrophoresis to separate based on their DNA fragment length. Since during electrophoresis, the shorter DNA fragment migrates first, the strengths of signals are measured over time using a measuring apparatus (1k) at a detection part (1j) to allow the signals corresponding to the existing nucleotides to be measured according to the nucleotide sequence in the target samples. A micro-channel developed using a technique called Micro-Electro-Mechanical Systems (MEMS) may be used to separate the labelled DNA fragments by electrophoresis and detecting the signals, in addition to the capillary-type flow channels.

The method for analyzing the DNA fragments using four separate flow channels is exemplified in FIG. 1. To improve further the accuracy of analysis, it is effective that the labeling reaction for each of four nucleotide types is induced with eight reactive solutions using two kinds of primers for one template to separate the labeled DNA fragments, which are the products of reactions, by electrophoresis for detection.

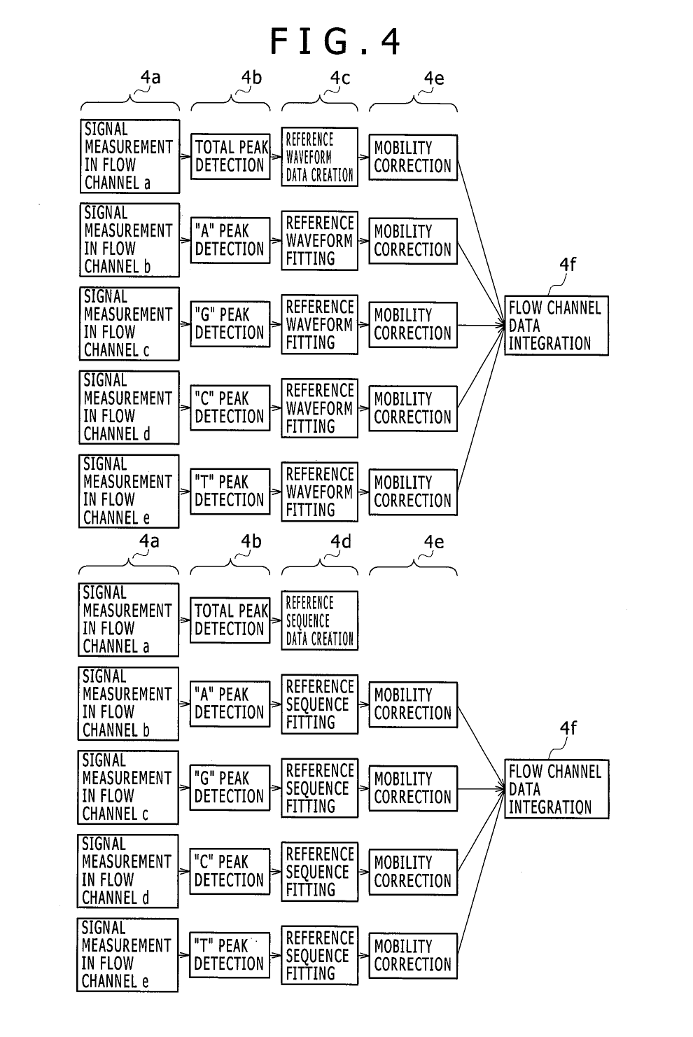

Moreover, for example, flow channels may be sequentially added depending on any other application, as explained later by reference to FIG. 4.

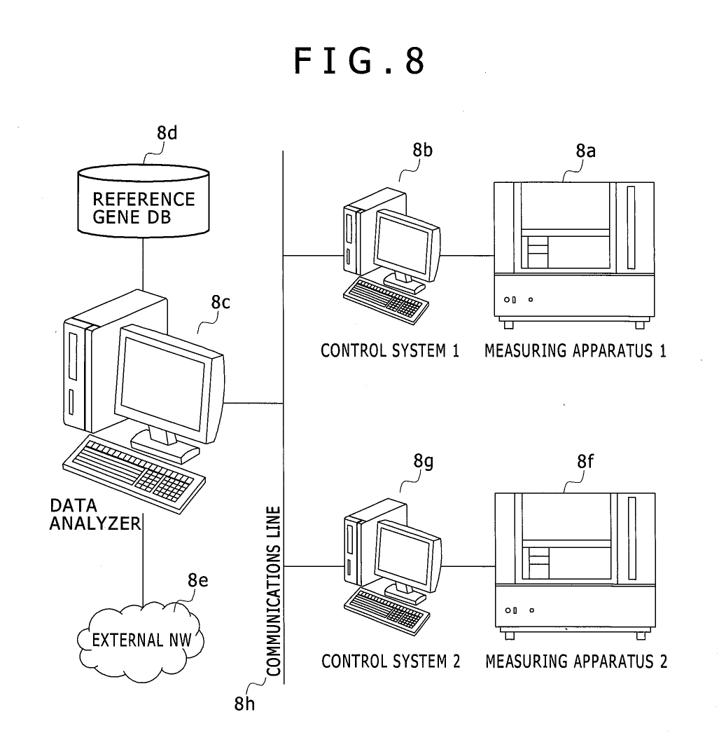

Next, an example of the configuration of the entire system according to the embodiment of the present invention will be explained by reference to FIG. 8. The system according to the embodiment of the present invention is composed mainly of a measuring apparatus (8a) for measuring the signal values by detecting the DNA fragments, as explained by reference to FIG. 1, and a data analyzer (8c) for correcting the signal values obtained from the measuring apparatus and displaying the results of data analysis. In this example, a control system (8b) connected to the measuring apparatus (8a), a reference gene database (8d), in which information on the target gene sequence is stored, and a data analyzer (8c) linked to an external network (8e) are connected via a communications line (8h), and additionally, the system may have a DNA detection function, a signal value correction function, and a result display function. Alternatively, the signal values obtained at the measuring apparatuses (8a) may be transmitted to an external computer connected thereto via the external network (8e) for performing the signal correction function. It should be noted that in the figure, control lines and information lines, which are considered to be necessary for explanation, are shown but all the control lines and information lines actually connected to the product are not always included. Actually, it may be considered that almost all the system components are connected to each other.

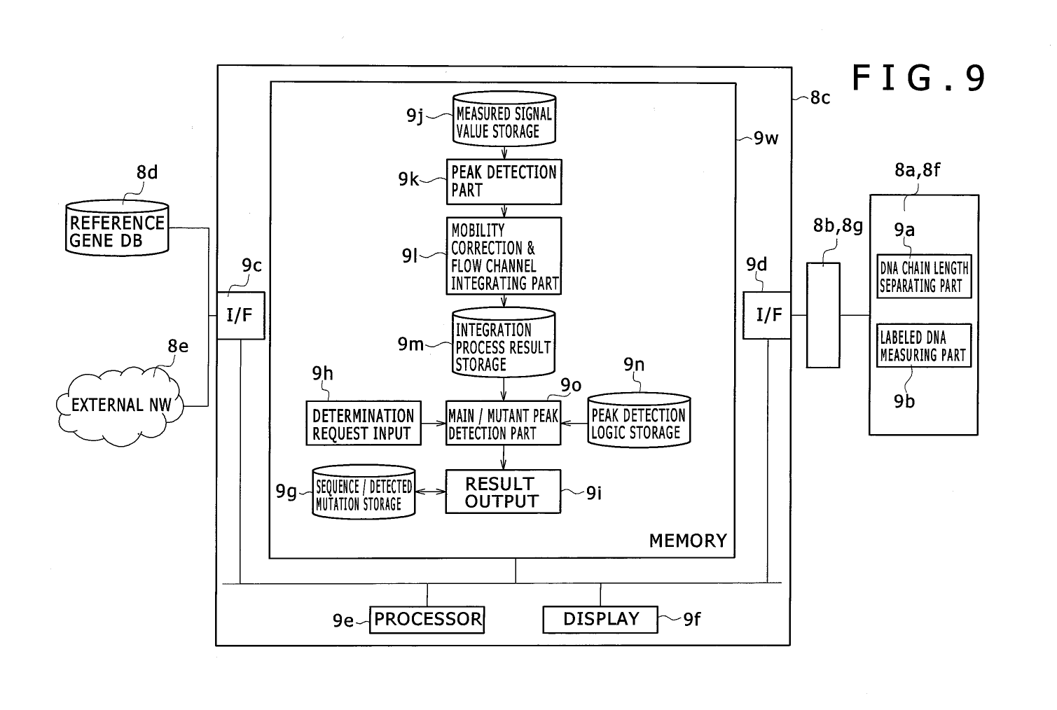

Next, the function parts contained in the data analyzer (8c) shown in FIG. 8 will be explained by reference to FIG. 9. To perform the functions at the function parts in the data analyzer (8c) shown in FIG. 9 by means of software, a processor (9e) interprets and executes a program for implementing the function, which is stored in memory (9f). Moreover, the data analyzer (8c) transmits information to and receives from an external device, the database, or the network via interfaces (9c,9d). Furthermore, the aforementioned configuration, function parts, processing parts, processing method, or the like may be implemented by means of hardware, for example, by designing totally or partially them using an integrated circuit. Information, such as the programs, files, and database for performing the functions, may be stored in recording devices, such as memory, hard disk, and solid state drive (SSD), or recording media, such as IC cards, SD cards, and DVD.

Initially, the measuring apparatuses (8a, 8f) separate DNA fragments based on their fragment length by electrophoresis, under the separation measurement conditions received from the control systems (8b, 8g), in the separate flow channel for each of nucleotide types shown in FIG. 1 at a DNA fragment length separation part (9a), and detects and measures the labeled DNA molecules at a labeled DNA measuring part (9b).

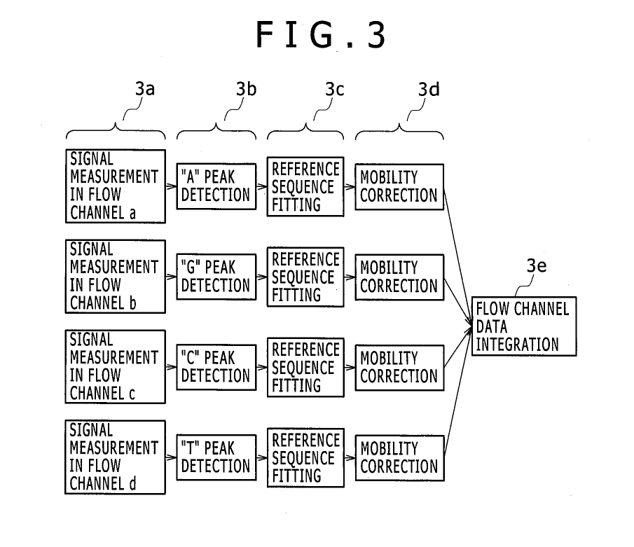

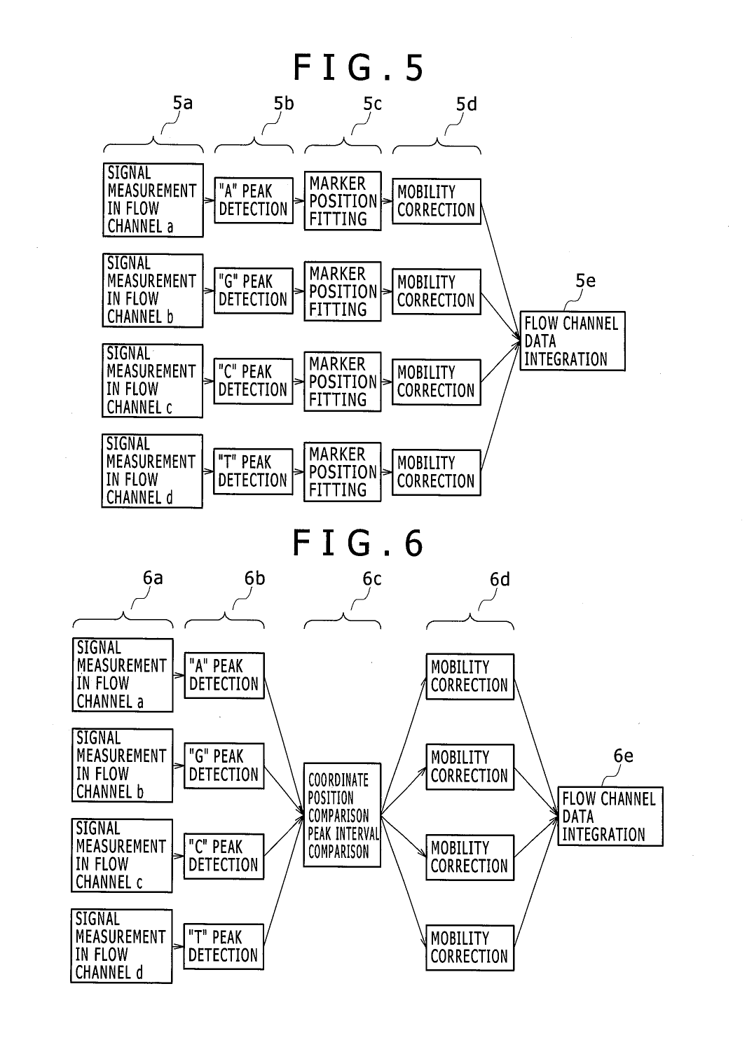

Then, the signal values measured at the measuring apparatus (8a) are transmitted to the data analyzer (8c). The data analyzer (8c) records the measured values, once received, in a measured signal value storage (9i). Then, using the method described later by reference to FIG. 3, FIG. 4, FIG. 5, and FIG. 6, a peak detection part (9k) detects peaks from the signal values stored in the measured signal value storage (9j) and a mobility correction & flow channel integrating part (9l) corrects the mobility among the measured results obtained from a plurality of flow channels corresponding the individual target samples, and integrates the data from the flow channels. Then, an integration process result storage (9m) records the results of integration. After that, a main/mutation peak detection part (9o) detects main peaks and mutation peaks based on the procedure for detecting the peaks recorded in a peak detection logic storage (9n) and a determination request issued by the user received from a determination request input (9h), such as the request for inputting any threshold value specified by a requester. In this case, the types of the nucleotides showing the signal values with strengths equal to or higher than the threshold value and the coordinates of them on the nucleotide sequence are extracted from the signal values stored in the integration process result storage (9m) to detect polymorphisms existing in the target analytical region exhaustively.

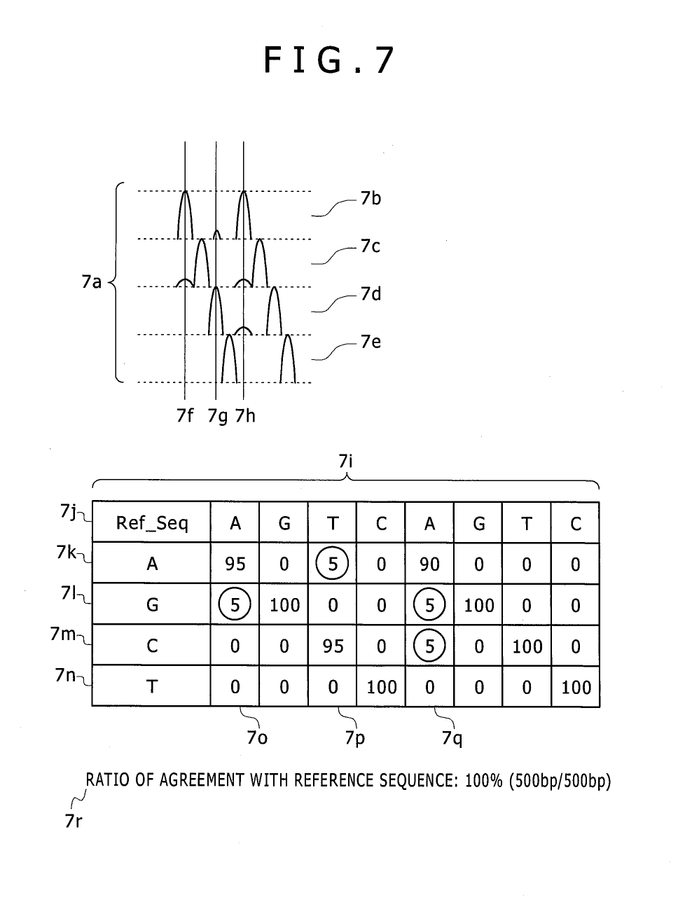

Then, a result output (9i) receives data detected at the main/mutation peak detection part (9o), records the data in a sequence/detected mutation storage (9g), and outputs the data to a display (9f). In turn, the display (9f) displays information on the nucleotide sequence and mutations, as well as the chromatograms, as described later by reference to FIG. 7. The sequence/detected mutation storage (9g) records the measured results to allow comparison analysis with the previously measured results to be performed and records the reference nucleotide sequence of the target gene to enable comparison analysis between the detected results and the reference sequence to be performed. The system is exemplified in the block diagram shown in FIG. 9; accordingly, the functions of the control system may be integrated with the functions of the data analyzer to execute on the same computer. Alternatively, as shown in FIG. 8, a plurality of measuring apparatuses may be linked to one data analyzer to analyze together the measured results obtained from the plurality of measuring apparatuses on one data analyzer.

Then, the peak detection function, which is performed at the peak detection part, (9k) described in FIG. 9 by reference to FIG. 3, FIG. 4, FIG. 5, and FIG. 6, and the function for correcting the mobility and integrating the data from the flow channels, which is performed at the mobility correction & flow channel integrating part (9l), will be explained. According to the embodiment of the present invention, the signal values (1l-1o), which are generated by one of nucleotide types labeled with its corresponding fluorescent substance, are detected in its corresponding flow channel because four types of nucleotides are analyzed in the separate flow channels; thereby, it is impossible to determine the gene sequence of the template DNA sample based on the signals obtained from each of the flow channels. To address this problem, a process (1p) of integrating the measured results obtained from the plurality of flow channels corresponding to the target samples is needed to calculate the target DNA measured results. It should be noted that since electrophoresis performed using the separate flow channels causes a difference in mobility, the function for correcting and integrating the mobility of the target measured is needed.

The methods for correcting the mobility using the signal values measured in each of the flow channels at the measuring apparatuses (8a, 8b), as input information, include; i) a method shown in FIG. 3, that corrects the mobility by comparing the appearance positions of each of nucleotide types based on known nucleotide sequence information or known reference chromatogram information in the target region with the positions, at which the signals with the strengths equal to or higher than the threshold value are measured, among those of the measured results of each of nucleotide types; ii) a method shown in FIG. 4, that corrects the mobility using at least either the reference chromatogram information or reference nucleotide sequence information on the target samples, as reference information, which is obtained by performing electrophoresis and analysis on four nucleotide types of the target samples in each of flow channels by the standard DNA sequencing method, in addition to the aforementioned measurement for each of nucleotide types; and iii) a method shown in FIG. 5, that corrects the mobility using the measured positions of DNA markers, as reference information, which is obtained by performing electrophoresis and measurement on the DNA samples labeled for each nucleotide type, to which the labeling substance with known fragment length has been added as the DNA marker; iv) a method shown in FIG. 6, that calculates the positions, at which signal strengths equal to or higher than the threshold are obtained, from the signal strengths for each of the nucleotide types obtained from each of the flow channels and corrects the mobility using, as an indicator, the property of continuous appearance, in each of the flow channels, of the signal values with the strengths equal to or higher than the threshold value, because the DNA molecules are polymeric molecules composed of continuous nucleotides; and v) a method, that performs the combination of two or more of the aforementioned methods i) to iv). Hereinafter, these methods will be explained.

The i) method shown in FIG. 3 involves the steps of; detecting the signal peaks for each of nucleotide types from the measured signal values (3a) obtained from each of flow channels at the measuring apparatuses (8a, 8b) (3b); performing fitting by comparing the appearance positions of each of nucleotide types based on the known nucleotide sequence information or known reference chromatogram information stored in the sequence/detected nutation storage (9g) with the information on the positions, at which the signals with the strengths equal to or higher than the threshold value are measured, from the signals measured for each of nucleotide types (3c); correcting the mobility based on the results of fitting (3d); and integrating the data obtained from each of the flow channels (3e).

The ii) method shown in FIG. 4 involves the steps of; performing electrophoresis and analysis on the four nucleotide types in their corresponding separate flow channels at the measuring apparatuses (8a, 8b) in the same manner as that of the sequencing method, in addition to measurement for each nucleotide type (4a); detecting the peaks for each nucleotide type from the measured signals (4a) from each of the flow channels and the peaks for all types of nucleotides from each of the flow channels (4b); performing fitting using at least either the reference chromatogram information or reference nucleotide sequence information on the target samples, as reference information, which is obtained based on the results of peak detection for all the fours nucleotide types from each of the flow channels (4c,4d); correcting and integrating the mobility based on the result of fitting (4e); and integrating the data from each of the flow channels (4f).

The method iii) shown in FIG. 5 involves the steps of; performing electrophoresis and measurement on the DNA samples, in each of which a labeled DNA molecule with known fragment length has been mixed as a DNA marker, at the measuring apparatuses (8a, 8b)(5a); detecting the peaks of signal values obtained from each of the flow channels in 5a for each of nucleotide types (5b); performing fitting using the positions of DNA marker measurement as reference information (5c); correcting the mobility based on the result of fitting; and integrating the data from each of the flow channels (5e).

The method iv) shown in FIG. 6 involves the steps of; detecting the peaks for each of nucleotide types from the signal values (6a) from each of the flow channels measured at the measuring apparatuses (8a, 8b)(6b); comparing among the positions and intervals of the peaks and calculating fitting conditions, under which the overlaps among the positions of detected peaks for four nucleotide types are minimum in size and the peak intervals are equalized (6c); correcting the mobility based on the result of fitting (6d); and integrating the data from each of the flow channels (6e).

As mentioned above, after the mobility of the measured signals of the target samples is corrected and the data is integrated, the types of the nucleotides showing the signals with the strengths equal to or higher than the threshold value and the coordinates of the signals on the nucleotide sequence are extracted through the process described by reference to FIG. 9 to detect the polymorphisms existing in the target region exhaustively. According to the embodiment of the present invention, cross talks due to fluorescent dyes, which cause a problem in the conventional DNA sequencers, are not found in the detected signal values, because separation and detection of each of nucleotide types are performed in the physically separate flow channels; accordingly, the existence of polymorphisms can be detected with a higher order of sensitivity than those of the conventional DNA sequencers.

Moreover, it is possible to calculate an index for the existence ratio of mutations using the largest one of and the smallest one of the signal values measured at the coordinate positions. In addition, to calculate the percent identity to reference sequence obtained from the target samples with the reference nucleotide sequence information, the nucleotide type showing the signal with the largest strength, among the signals, with strengths equal to or higher than the threshold value, which are obtained from the target samples, may be determined to compare with the information on the known reference nucleotide sequence in the target region.

Next, an example of the method for displaying the results of analysis according to the embodiment of the present invention will be explained by reference to FIG. 7. In the figure, 7a shows the exemplified integrated information from each of the flow channels after the mobility has been corrected, including the information on the positions, at which the signals of each nucleotide type and the information on the strengths of the detected signals (7b-7e). A weak signal with the strength equal to or higher than the threshold value, which is detected at the same abscissa position after the mobility has been corrected for each of the flow channels, indicates the existence of a polymorphism at the position of the nucleotide. In the figure, 7f to 7h show the example of the coordinate positions, at which the existences of polymorphisms have been detected.

In the figure, 7i shows an exemplified display of the list of the results of exhaustive detection of the polymorphisms existing in the target region, based on the measured results obtained by integrating data from each of the flow channels (7a). As shown in 7i, the existence ratios of four nucleotide types showing the signals with the strengths higher than the threshold value among the calculated ones are displayed in the form of a list by plotting information on the reference nucleotide sequence along the abscissa axis and the nucleotide types along the ordinate axis. As shown in 7f-7h of the integrated chromatogram information (7a), the signals detected along the same abscissa axis show the existences of the polymorphisms at the positions of the nucleotides corresponding to the signals; accordingly, it is possible to display the existence ratios of polymorphisms, which are calculated by comparing the nucleotide types of existing polymorphisms and the strengths of the signals at the same coordinate positions (7o-7q). In addition, the percent identity of the nucleotide sequence obtained from target samples with information on the reference nucleotide sequence can be calculated by determining the nucleotide type showing the signal with the largest strength among the strengths equal to or higher than the threshold value at the coordinate positions, which are obtained from the target samples, and comparing the determined nucleotide type with the known information on the reference nucleotide sequence in the target region (7r). The percent identity with the reference nucleotide sequence information plays a preferable role as an indicator for determining whether the measured DNA region agrees with the intended target region to be measured.

Alternatively, in addition to displaying the results of measurement as shown in 7i, the system may be configured so that i) the positions, at which polymorphisms are detected, are extracted or enhanced for displaying; ii) the positions of known polymorphisms related to diseases are extracted or enhanced for display; or iii) the specific coordinate positions specified by a tester are extracted or enhanced for display. The exhaustive information on polymorphisms obtained from the target region shows the existences of minute mutations in somatic cells, which cannot be acquired by the existing DNA sequencers and quantitative PCR apparatuses; accordingly, it is useful in analyzing the correlation between the mutations in somatic cells and diseases and treating these diseases.

If the existences of polymorphisms in the target gene region and the existence ratios thereof serve as indicators for medicine administration or medical treatment, the aforementioned results of exhaustive polymorphism detection may be displayed effectively on the analyzers for medical use because the guideline for whether these medicines can be administered, the dosages of these medicines, and the medical treatment is directly provided. Moreover, the results of exhaustive polymorphism detection may be compared with other clinical information and the outcomes from therapy to work out further effective medicine regimens.

Furthermore, a kit for analyzing genes, supplied with reagents for gene mutation detection, which label the nucleic acid samples for each of nucleotide types, may be used to detect genetic mutations using the aforementioned system; the kit performs electrophoresis in the separate channel for each of nucleotide types and detects genetic mutations for each of the nucleic acid samples based on the chromatogram data obtained from the labeled signal for each of nucleotide types from its corresponding separate flow channel.

Thus, according to the embodiment of the present invention, performing electrophoresis and detection for each of nucleotide types using the separate flow channels allows the polymorphisms in the somatic cell existing in the target gene region to be detected with a high order of sensitivity. Additionally, comparison among the detected strengths of signals enables the existence ratios of the mutations in somatic cell to be analyzed. The obtained exhaustive information on polymorphisms in the target region shows the existences of minute mutations in somatic cells, which cannot be acquired by the existing DNA sequencers or quantitative PCR systems; it is useful in analyzing the correlation between the mutations in somatic cells and diseases and medical treatment thereof. Moreover, comparison of the obtained information on the polymorphisms in the somatic cell line, against the existing information on the polymorphisms in the somatic cell line, clinical information, and the outcomes from therapy enables further effective medicine regimens to be worked out.

1. A system for analyzing genes comprising:
a plurality of flow channels that perform electrophoresis on nucleic acid samples, in which each of nucleotide types has been labeled, individually for each of the nucleotide types;
chromatogram data creating part that detects a labeled signal for each of the nucleotide types for each of the nucleic acid samples electrophoresed in each of the flow channels and creates the chromatogram data on the strengths of the detected signals;
a detection part that detects the peak values in the chromatogram data for each of the nucleotide types;
a data integrating part that integrates the plurality of chromatogram data, in which the peak values have been detected; and
a display that displays the integrated data.

2. The system for analyzing genes according to claim 1, wherein the data integrating part corrects a difference in mobility of the samples among the plurality of flow channels during electrophoresis and integrates data from the plurality of flow channels.

3. The system for analyzing genes according to claim 1, wherein a polymorphism analyzing part is further contained for comparing among the peak values measured in each of the plurality of flow channels to calculate the existence ratio of gene polymorphism coordinate positions on the same nucleotide sequence after the integration process, and the display displays the calculated results at the polymorphism analyzing part.

4. The system for analyzing genes according to claim 3, wherein the polymorphism analyzing part calculates the percent identity to reference sequence of the information on base nucleotide sequence obtained from the nucleic acid samples with the information on reference nucleotide sequence, based on the existence ratio of polymorphisms and the result of comparison with the known information on reference nucleotide sequence.

5. The system for analyzing genes according to claim 1, wherein the peak detection part extracts the signal strengths equal to or higher than the given threshold value, as the peak values, from the chromatogram data for each of the nucleotide types.

6. A method for analyzing genes comprising:
separating the nucleic acid samples, in which each of nucleotide types has been labeled, in a separate flow channel for each nucleotide type by electrophoresis;
detecting labeled signals for each of nucleotide types of each of the nucleic acid samples electrophoresed in each of the plurality of flow channels to create chromatogram data;
detecting the peak values in the chromatogram data for each of the nucleotide types;
integrating the plurality of chromatogram data, in which the peak values have been detected; and
displaying the integrated data.

7. The method for analyzing genes according to claim 6, wherein differences in mobility of the samples among the flow channels generated during the electrophoresis.

8. The method for analyzing genes according to claim 6, after integrating the data, further comprising:
comparing among the peaks values measured for each of the plurality of flow channels;
calculating the existence ratios of gene polymorphisms at the coordinate positions on the same nucleotide sequence; and
displaying the results of calculation obtained at the polymorphism analyzing part.

9. The method for analyzing genes according to claim 8, wherein based on the result of comparison between the existence ratios of the gene polymorphisms and known information on reference nucleotide sequence, the ratios of agreement of information on nucleotide sequence obtained from the nucleic acid samples with the known information on reference nucleotide sequence is calculated.

10. The method for analyzing genes according to claim 6, wherein signal strengths equal to or higher than the given threshold value are extracted from the chromatogram data for each of the base types.

11. A kit for analyzing genes with reagents for detecting genetic mutations, which label individually four nucleotide types, comprising:
labeling nucleic acid samples for each of the nucleotide types;
electrophoresing the samples in a separate flow channel for each of the Nucleotide types; and
detecting genetic mutations for each of the nucleic acid samples electrophoresed in each of the plurality of flow channels in the previous process based on chromatogram data obtained from a labeled signal for each of the nucleotide types.
